(19) United States
(12) Patent Application Publication
(10) Pub. No.: US 2016/0278965 A1
(43) Pub. Date: Sep. 29, 2016

(54) Medical Fastening Device

(71) Applicants: Kálmán BOLLA, Budapest (HU); István FALUSI, Budapest (HU)

(72) Inventors: Kálmán BOLLA, Budapest (HU); István FALUSI, Budapest (HU)

(73) Assignee: ORKRISZ KFT., Budapest (HU)

(21) Appl. No.: 14/670,739

(22) Filed: Mar. 27, 2015

(51) Int. Cl. A61F 5/058

(52) U.S. Cl. CPC A61F 5/05825

(57) ABSTRACT

Medical immobilizing device comprising a single waved sheet member (10), wherein ribs and channels which form the waves of said sheet member are arranged in parallel, wherein the sheet member (10) comprises at least two flexible zones (A, B), wherein the waves of different zones have different orientation.

The invention relates to a medical immobilizing device comprising a single waved sheet member, wherein ribs and channels which form the waves of said sheet member are arranged in parallel.

The application of splints for body parts, limbs or the circular stable fixation (by plaster of Paris) thereof for therapeutic purposes are known in the art. In case of medical splints the stable fixation is limited only to a band or bands depending on the width of the splint, which is/are secured to the limb with textile straps or a wrap. This fixation prohibits the movement perfectly only in one specific direction, which depends on the sheet of the splint.

Formerly, fixing devices were used that could be fitted to the injured body part flexibly, but only along a band, i.e. along one dimension. In their given position, these fixing means then provided adequate mechanical stability. Among others WO 97/22312 discloses a semi-circularly stable fixing device, namely a medical splint, which contains a waved sheet, wherein due to the waving being perpendicular to the axis of the limb, the splint, that provides the stability, can be bent around the body part. The base sheet, which is waved in a direction transverse to the longitudinal axis of the limb is then furnished with a further sheet of smaller area, having waves in a different direction so that in this smaller area it provides a stronger support/fixation for the limb part covered by the second sheet than in other areas of the fixing device. This further sheet, however, isn't intended to fix (to splint) the injured limb part, but it functions as a supplementary support.

A drawback of this solution is that the spatial deformability and rigidity of the sheet for fixing the injured limb is largely limited due to the waves being arranged in a single direction, furthermore it cannot ensure the fixation of the limb parts with various strengths without material surplus and the increased thickness at the areas of the further sheets.

Therefore the objectives of the present invention are to provide a medical immobilizing device, which meets the requirements of differentiated fixation and the convenience of the patient more, whereas the immobilizing device has sufficient rigidity along the entire fixing area of the injured body part.

These objectives are reached by providing a medical immobilizing device, which comprises a single waved sheet member, wherein ribs and channels which form the waves of said sheet member are arranged in parallel, furthermore the sheet member comprises at least two flexible zones, wherein the waves of different zones have different orientation.

Preferably the angle between the orientation of the waves of a first zone and the orientation of the waves of a second zone form an acute angle.

Preferably the orientation of the waves of a first zone is perpendicular to the orientation of the waves of a second zone.

The device preferably comprises three zones, each having a different orientation of waves.

Preferably, one of the zones is formed entirely within another zone.

The sheet member is preferably provided with an elastic cover.

Preferably the cover is provided with a securing means for securing to a body part to be immobilized.

Preferably the sheet member is formed as an aluminum sheet having a thickness of 0.2-1.1 mm.

Preferably the height of the waves forming the waves of said sheet member ranges from 1.0 mm to 2.5 mm.

The invention will now be further described with reference to the following embodiments and drawings in which

FIG. 1 illustrates an unfolded view of the sheet member of the immobilizing device for immobilizing the carpometacarpal articulation of a thumb,

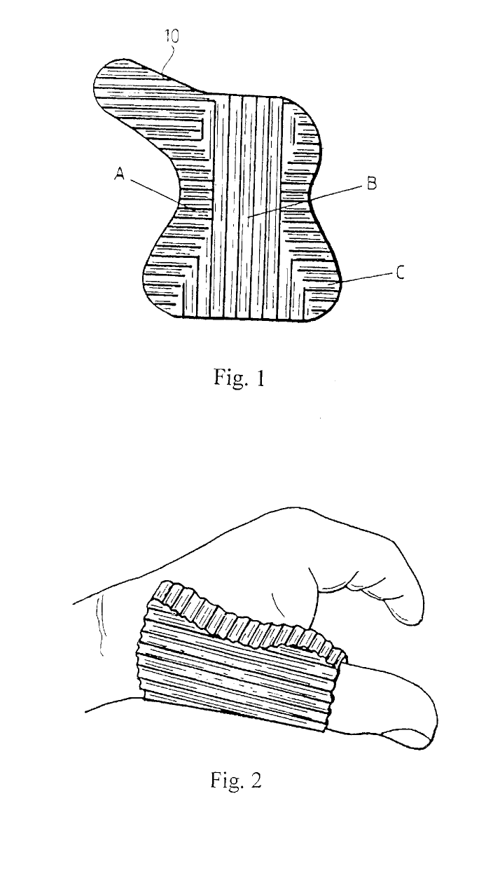

FIG. 2 depicts the sheet member of FIG. 1 in an applied state,

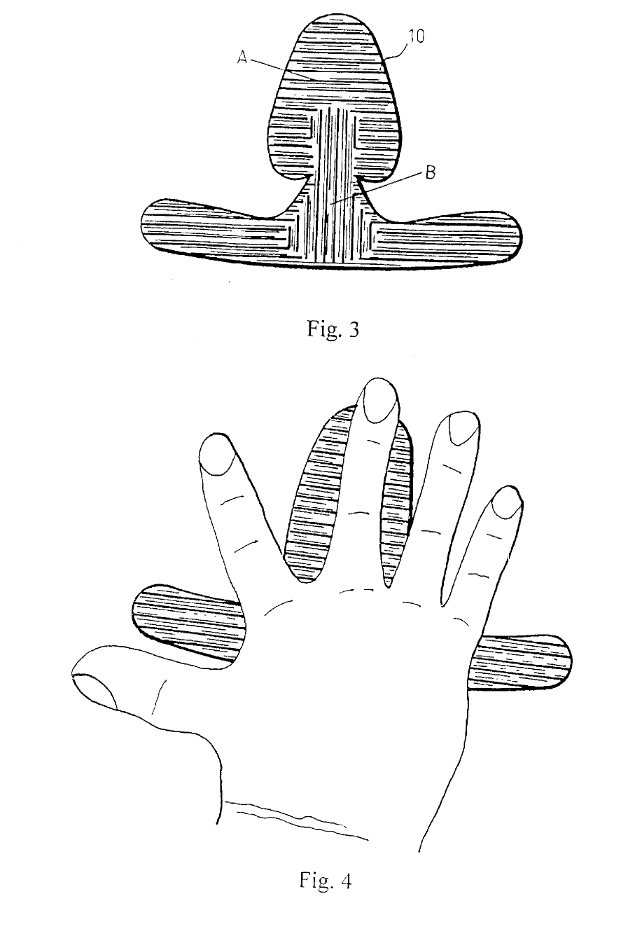

FIG. 3 shows an unfolded view of the sheet member of the immobilizing device for immobilizing the middle finger,

FIG. 4 shows the sheet member of the device of FIG. 3 put under the middle finger

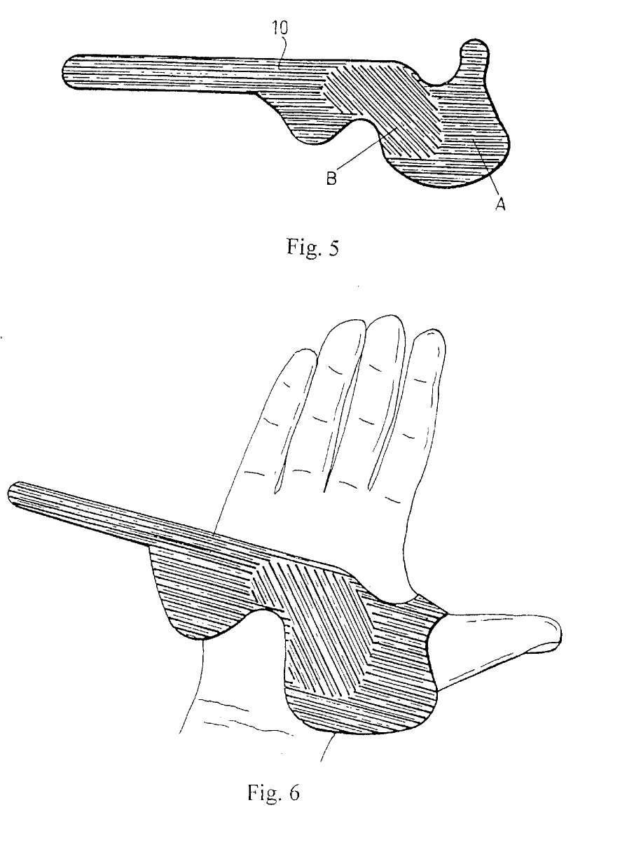

FIG. 5 illustrates an unfolded view of the sheet member of an immobilizing element usable as a short splint for a saddle articulation,

FIG. 6 shows the sheet member of the immobilizing device of FIG. 5 applied on the palm,

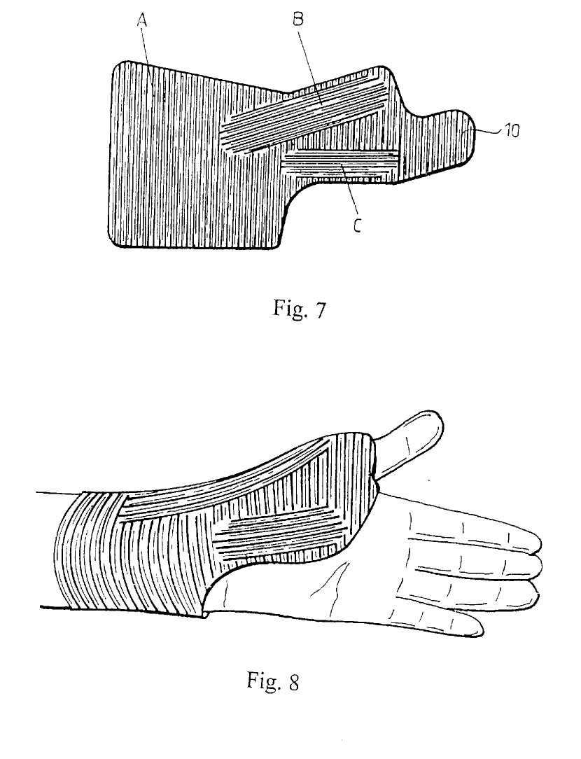

FIG. 7 shows an unfolded view of the sheet member of an immobilizing element usable as a functional saddle joint splint, and

FIG. 8 depicts the sheet member of the immobilizing device of FIG. 7 during use.

The immobilizing device shown in FIGS. 1 and 2 serves to fix the carpometacarpal articulation of a thumb with various strengths. As shown in the unfolded view in FIG. 1, the sheet member 10 of the immobilizing device of the present invention comprises three A, B, C zones, wherein the orientation of the waves of zones A and C is identical, while zone C has waves of a different orientation with respect to those of A and C, in the present case, perpendicular thereto. As shown in FIG. 2, the first orientation of the waves of zones A and C ensures that the immobilizing device can follow the contour of the thumb and the hand, furthermore these zones provide sufficient rigidity for the entire thumb. The second orientation (which is perpendicular to the first orientation in this embodiment) waves formed in zone B, however, hinder the flexing of the articulation under zone B. As can be seen in FIG. 2, the shape of the sheet member 10 can be perfectly fitted to the spatial shape of the injured body part, while by means of the suitable waves, the zones due to their rigidity deriving from the orientation of the waves, prohibit any movement of the injured body part.

FIGS. 3 and 4 show an immobilizing device for immobilizing a middle finger. As can be seen in FIG. 3, the sheet member of the immobilizing device of the present invention, in this case contains two flexible A and B zones, the waves of which have different orientation. In this embodiment, the orientation of the waves of zones A and B are also perpendicular to each other. As is shown in FIG. 4, the sheet area of zone A provided with waves of a first orientation immobilizes the middle finger in such a way that it substantially surrounds the middle finger, while due to the orientation of zone B being different, it hinders the flexing of the finger to be immobilized.

FIGS. 5 and 6 show an immobilizing device that can be used as a short splint for a saddle articulation. The sheet member 10 of the immobilizing device of the present invention contains two flexible A and B zones in this embodiment as well, wherein zone B is formed entirely within zone A. In this embodiment, however, the waves of zones A and B are not perpendicular, but form an angle of about 45 degrees. Due to this configuration, the sheet member 10 immobilizes the finger such that it hinders the flexing of the carpometacarpal articulation of the thumb (MCP), at the same time, by means of fitting the sheet member 10 around the palm, it prevents the movement of the thumb in other directions (e.g. flexion, extension, adduction, abduction, opposition and circumdiction).

FIGS. 7 and 8 show an immobilizing device that can be used as a functional splint for a saddle articulation. In this embodiment, the sheet member 10 of the immobilizing device of the present invention comprises three, A, B and C flexible zones, wherein zones B and C are formed within zone A, and the waves of different zones have different orientation. As can be seen in FIG. 7, in this embodiment the waves of zone B form an angle of 60 degrees, whereas the waves of zone C form an angle of 90 degrees with the waves of zone A, respectively. Zone A, due to its fitting around the wrist or the lower arm, prevents the movement of the entire thumb, moreover zones B and C ensure increased rigidity by zone B providing support for the thumb against flexing at the upper side of the thumb and by zone C preventing the so-called oppositional movement of the saddle articulation of the thumb.

The above-presented two types of the immobilizing device for saddle articulation (i.e. the third embodiment shown in FIGS. 5 and 6 and the fourth embodiment shown in FIGS. 7 and 8) differ in that while the third embodiment mainly aims at the best possible immobilization and is recommended to be used as a resting splint, then the functional fourth embodiment—according to its name—assists in the painless carrying out of the usual daytime activities so that it only partly hinders the movement within the saddle articulation.

Preferably, the sheet member 10 of the immobilizing device of the present invention is formed as an aluminum sheet having a thickness of 0.2 to 1.1 mm, wherein the sheet is provided with waves of adequate height and width, e.g. by sheet-metal pressing. The waved sheet member 10 can also be made of other material (e.g. of plastic), which can be elastically deformable, while ensures sufficient flexural rigidity for the immobilization of the injured body part at the desired level. The height of the waives of the sheet member 10 is preferably 1.0-2.5 mm depending on the location of the injured body part and and the probable loading of the immobilization device. The deformability of the zones can also be influenced by the width of the waves; the broader the waves are, the more flexible the sheet member 10 is.

The presented examples show that the medical immobilizing device of the present invention ensures the immobilization of the various limb and trunk articulations much more easily and efficiently than the previous solutions. Although in the presented examples we only illustrated the different orientations of the waves, it is apparent that for the immobilization of the articulations, and also for the application of the immobilization device itself, as well as for meeting the needs, not only the orientation of the waves, but also other parameters thereof, such as the height or width of the waves, can be different in the different zones, furthermore in case of need, waves of changing height and/or width can be formed within the individual zones depending on their distances from the borders of the given zones.

For the sake of simplicity, the drawings show only various exemplary versions of the sheet member of the immobilizing device of the present invention. It is nonetheless obvious for a skilled person that at the medical immobilizing devices according to the present invention, although the sheet member itself can function as an immobilizing device independently, because of comfort, hygienic or other reasons, the sheet member can be provided with an outer cover, e.g. an elastic textile wrap, moreover for the enhanced securing of the immobilizing device to the given body part, it can be provided with other known securing means (e.g. a hook and loop strip). All these supplementary elements are well known in the art, therefore their description is omitted here.

The presented embodiments are merely intended to illustrate the invention and it is obvious that a person skilled in the art could devise numerous other solutions within the scope of the attached claims concerning both the upper and the lower limb, and even the trunk.

1. Medical immobilizing device comprising a single waved sheet member (10), wherein ribs and channels which form the waves of said sheet member are arranged in parallel, characterized in that the sheet member (10) comprises at least two flexible zones (A, B), wherein the waves of different zones have different orientation.

2. The device according to claim 1, characterized in that the angle between the orientation of the waves of a first zone (A) and the orientation of the waves of a second zone (B) form an acute angle.

3. The device according to claim 1, characterized in that the orientation of the waves of a first zone (A) is perpendicular to the orientation of the waves of a second zone (B).

4. The device according to claim 1, characterized in that the device comprises three zones (A, B, C), each having a a different orientation of waves.

5. The device according to claim 1, characterized in that one of the zones (B) is formed entirely within another zone (A).

6. The device according to claim 1, characterized in that the sheet member is provided with an elastic cover.

7. The device according to claim 6, characterized in that the cover is provided with a securing means for securing to a body part to be immobilized.

8. The device according to claim 1, characterized in that the sheet member is formed as an aluminum sheet having a thickness of 0.2-1.1 mm.

9. The device according to claim 1, characterized in that the height of the waves forming the waves of said sheet member ranges from 1.0 mm to 2.5 mm.
